(19) United States
(12) Patent Application Publication
(10) Pub. No.: US 2025/0134604 A1
(43) Pub. Date: May 1, 2025

(54) ASSISTED SURGERY SYSTEM IN RADIOTHERAPY OR ABLATION THERAPY PROCESS AND SURGICAL ROBOT

(71) Applicant: Yong Jiang, Guangdong (CN)

(72) Inventor: Yong Jiang, Guangdong (CN)

(21) Appl. No.: 18/836,467

(22) PCT Filed: Jul. 21, 2022
(86) PCT No.: PCT/CN2022/107027

§ 371 Date: Aug. 7, 2024

(30) Foreign Application Priority Data
Jul. 19, 2022 (CN) 202210845742.9

(51) Int. Cl. A61B 34/30; A61B 1/00

(52) U.S. Cl. CPC A61B 34/30; A61B 1/00045; A61B 1/00082; A61B 2034/301

(57) ABSTRACT

The present disclosure belongs to the technical field of assisted medical instruments, and in particular, to a surgery assisting system in radiotherapy or ablation therapy process and a surgical robot. Specifically, the inflation amount of the organ isolation balloon is configured by acquiring the morphological information about the target organ to be subjected to radiotherapy or ablation therapy and the effect information required to be achieved by the treatment. Then the organ isolation balloon is moved to the target position by the delivering device. The organ isolation balloon is inflated via the airflow control component such that the inflated organ isolation balloon adapts to the target organ. The organ isolation balloon is then fixed to the body wall by a fixing device for treatment guidance.

### TECHNICAL FIELD

The present disclosure relates to the technical field of assisted surgical medical instruments, and in particular, to a surgery assisting system in radiotherapy or ablation therapy process and a surgical robot.

### BACKGROUND

In some clinical treatments, methods such as radiotherapy and ablation therapy are often used to treat diseased organs. For example, in clinical practice, radiofrequency ablation therapy is used to achieve minimally invasive treatment of solid tumors. For radiofrequency ablation therapy; each ablation can achieve a maximum diameter of 4.5 to 6.0 cm per site (single-stage or multi-stage), with minimal impact on the surrounding organ tissue structures. Radiofrequency ablation therapy has the advantages of being minimally invasive, effective, and having few adverse reactions.

However, radiotherapy and ablation therapy may cause adverse effects on the adjacent tissues around the target area. In practical application, concerns about damaging the surrounding tissues, which often lead to and increase corresponding complications, result in insufficient ablation therapy area, incomplete treatment or tumor recurrence.

In this regard, CN1915184A discloses an ablation therapy isolation balloon. This balloon uses a hollow tube with a balloon material that is implanted into the patient's body through a catheter sheath. By inflating or filling the balloon material with air or water, it expands and creates a barrier between the ablation treatment site and the adjacent tissues and organs, thereby reducing damage to other organs and tissues adjacent to the lesion and lowering the incidence of complications from ablation therapy: The patent application provides a switch at the end of the hollow tube which, in use, opens to inject air into the balloon material to inflate the balloon material. However, the balloon material in the patent application expands unevenly due to natural inflation, is prone to air leakage after inflation, has a short isolation time, and cannot effectively ensure the isolation of all organs, resulting in poor repeatability. In addition, the balloon material of the patent application cannot be used once it is damaged, and thus has poor reliability. Moreover, in the patent application, the rear end of the balloon material is fixed by the hollow tube, while the front end of the balloon material cannot be fixed. Coupled with the movement of human organs within the body, this structure of the balloon cannot completely isolate the organ to be treated from other adjacent organs or tissues.

### SUMMARY

In view of this, embodiments of the present disclosure provide a surgery assisting system in radiotherapy or ablation therapy process and a surgical robot that addresses the technical problem of poor isolation of existing isolation balloons, which cannot be used once they are damaged.

The technical solutions used by the present disclosure are as follows.

In a first aspect, the present disclosure provides a surgery assisting system in radiotherapy or ablation therapy process, the surgery assisting system including a control device, an organ isolation balloon, a delivering device, an inflation device and a fixing device, the control device being configured for:
acquiring morphological information about a target organ to be subjected to radiotherapy or ablation therapy and effect information corresponding to the radiotherapy or ablation therapy;
configuring an inflation amount of the uninflated organ isolation balloon based on the morphological information and the effect information to obtain inflation configuration information; moving, by the delivering device, the uninflated organ isolation balloon to a target position that is set based on the target organ and the effect information;
inflating, by the inflation device, the organ isolation balloon based on the inflation configuration information and using an airflow control component configured in the organ isolation balloon, such that the inflated organ isolation balloon adapts to the target organ; and
fixing, by the fixing device, the inflated organ isolation balloon onto a body wall around the target position to obtain guide information for indicating progress of the radiotherapy or ablation therapy.

Optionally, in some possible implementations of the present disclosure, the organ isolation balloon includes a plurality of balloon units, and the configuring an inflation amount of the uninflated organ isolation balloon based on the morphological information and the effect information to obtain inflation configuration information includes:
determining acting range information corresponding to a position requiring protection or isolation in radiotherapy or ablation therapy process based on the morphological information;
configuring a quantity of the balloon units according to the acting range information corresponding to a position requiring protection or isolation such that the organ isolation balloon covers an acting range on the target organ corresponding to the acting range information;
connecting the balloon units according to positions of splicing sites at edges of the balloon units based on the quantity of the balloon units to obtain the organ isolation balloon; and
configuring a unit inflation amount of each of the balloon units in the organ isolation balloon according to the effect information to obtain the inflation configuration information.

Optionally, in some possible implementations of the present disclosure, the configuring a unit inflation amount of each of the balloon units in the organ isolation balloon according to the effect information to obtain the inflation configuration information includes:
acquiring the effect information acting on the target organ in radiotherapy or ablation therapy process, and determining an acting dose required to reach the effect information, where the acting dose corresponds to a dose when the radiotherapy is about to endanger an adjacent organ or a dose that is capable of protecting an adjacent organ in the ablation therapy;
configuring an isolation distance for achieving an isolation effect on the target organ according to the acting dose;
configuring a unit inflation amount of each of the balloon units in the organ isolation balloon based on the isolation distance to obtain the inflation configuration information.

Optionally, in some possible implementations of the present disclosure, the configuring a unit inflation amount of each of the balloon units in the organ isolation balloon according to the effect information to obtain the inflation configuration information includes:
acquiring the effect information acting on the target organ in radiotherapy or ablation therapy process, and determining edge state information corresponding to the effect information on the target organ;
configuring an inflation amount gradient attached to the target organ based on the edge state information about the target organ; and
determining the unit inflation amount of each of the balloon units in the organ isolation balloon according to the inflation amount gradient to obtain the inflation configuration information.

Optionally, in some possible implementations of the present disclosure, the organ isolation balloon includes a first isolation balloon block and a second isolation balloon block, and
the moving, by the delivering device, the uninflated organ isolation balloon to a target position includes:
   determining splicing information about the first isolation balloon block and the second isolation balloon block corresponding to the organ isolation balloon in radiotherapy or ablation therapy process based on the morphological information; and
   determining a first position to which the first isolation balloon block needs to be moved and a second position to which the second isolation balloon block needs to be moved according to a target position configured for the organ isolation balloon and the splicing information, the second position being associated with the first position through the target position;
   moving, by the delivering device, the first isolation balloon block and the second isolation balloon block such that the first isolation balloon block moves to the first position and the second isolation balloon block moves to the second position:
accordingly, the inflating, by the inflation device, the organ isolation balloon based on the inflation configuration information and using an airflow control unit configured in the organ isolation balloon such that the inflated organ isolation balloon adapts to the target organ includes:
   inflating, by the inflation device, the first isolation balloon block and the second isolation balloon block using the inflation configuration information;
   determining a first splicing site at an edge of the inflated first isolation balloon block and a second splicing site at an edge of the inflated second isolation balloon block according to the splicing information; and
   splicing the edge of the inflated first isolation balloon block and the edge of the inflated second isolation balloon block based on the first splicing site and the second splicing site such that the inflated first isolation balloon block and the inflated second isolation balloon block wrap the target organ.

Optionally, in some possible implementations of the present disclosure, the moving, by the delivering device, the first isolation balloon block and the second isolation balloon block such that the first isolation balloon block moves to the first position and the second isolation balloon block moves to the second position includes:
determining an adjacent target object corresponding to the target organ and requiring protection;
planning a first path for the first isolation balloon block based on a position of the target object such that the first isolation balloon block bypasses the target object during movement;
delivering, by the delivering device, the first isolation balloon block to the first position using the first path through a delivery hole;
planning a second path for the second isolation balloon block based on the position of the adjacent organ such that the second isolation balloon block bypasses a target object during movement: and
delivering, by the delivering device, the second isolation balloon block to the second position using the second path through the delivery hole.

Optionally, in some possible implementations of the present disclosure, the delivering device includes a delivery conduit and a balloon delivery member:
the delivery conduit is provided with a hollow pipeline, the balloon delivery member is provided with a storage groove for storing the organ isolation balloon at one end of the balloon delivery member, the balloon delivery member is delivered to the target position inside a human body along the pipeline of the delivery conduit, and the storage groove is exposed out of the delivery conduit; and
the delivery conduit is further configured for delivering a puncture needle, where a tip of the puncture needle penetrates from one end of the delivery conduit to pierce the inflated organ isolation balloon and penetrates from the other end of the delivery conduit, and the pierced organ isolation balloon is taken out of the delivery conduit.

Optionally, in some possible implementations of the present disclosure, the organ isolation balloon in the surgery assisting system includes a plurality of balloon units, where each of the balloon units is provided with a corresponding airflow control component for communicating an interior of a chamber of the respective balloon unit with the inflation device for providing air when filling the air into the chamber, such that the air passes from outside of the chamber of the balloon unit into the chamber of the balloon unit and is prevented from leaking out of the chamber of the balloon unit.

Optionally, in some possible implementations of the application, the surgery system further includes an endoscope and a television monitoring device, the endoscope being configured for acquiring real-time images of an interior of the human body and transmitting the real-time images to the television monitoring device, and the endoscope being configured for displaying the morphological information, the inflation configuration information, and the guide information in radiotherapy or ablation therapy process.

In a second aspect, the present disclosure provides a surgical robot including a control circuit, a manipulator and the surgery assisting system according to any of the above implementations, the control circuit being electrically connected to the endoscope and the control circuit, respectively, where the control circuit is configured for receiving real-time images of an interior of a human body captured by the endoscope, controlling the manipulator to convey a balloon delivery member with the organ isolation balloon along a pipeline of a delivery conduit to a target position inside the human body according to the real-time images, and controlling the inflation device to inflate the balloon after the balloon delivery member is delivered to the target position within the human body.

In conjunction with the above possible implementations, it can be seen that the inflation amount of the organ isolation balloon is configured by acquiring the morphological information about the target organ to be subjected to radiotherapy or ablation therapy and the effect information required to be achieved by the treatment. Then the organ isolation balloon is moved to the target position by the delivering device. The organ isolation balloon is inflated via the airflow control component such that the inflated organ isolation balloon adapts to the target organ. The organ isolation balloon is then fixed to the body wall by a fixing device for treatment guidance. Due to the targeted configuration for the organ isolation balloon based on the morphology of the target organ, the effective degree of the organ isolation balloon for the protection of the target organ in radiotherapy or ablation therapy process is improved, and the negative impact of the radiotherapy or ablation therapy on the adjacent organs is reduced.

### BRIEF DESCRIPTION OF THE DRAWINGS

In order to explain the technical solution of the embodiments of the present disclosure more clearly, the following brief description will be given to the accompanying drawings which are used in the embodiments of the present disclosure, and it would have been apparent for a person of ordinary skill in the art to obtain other drawings according to the present disclosure without involving any inventive effort, and these are all within the scope of the present disclosure.

FIG. 1 is a schematic diagram showing the framework of a surgery assisting system according to an embodiment of the present disclosure:

FIG. 2 is a schematic flow chart showing implementation of a surgery assisting system according to an embodiment of the present disclosure:

FIG. 3 is a schematic structural diagram showing an organ isolation balloon according to an embodiment of the present disclosure:

FIG. 4 is a schematic structural diagram showing a balloon unit according to an embodiment of the present disclosure:

FIG. 5 is a schematic structural diagram showing another organ isolation balloon according to an embodiment of the present disclosure:

FIG. 6 is a schematic structural diagram showing another check valve according to an embodiment of the present disclosure:

FIG. 7 is a schematic structural diagram showing another organ isolation balloon according to an embodiment of the present disclosure:

FIG. 8 is a schematic structural diagram showing another organ isolation balloon according to an embodiment of the present disclosure:

FIG. 9 is a schematic structural diagram showing another organ isolation balloon according to an embodiment of the present disclosure:

FIG. 10 is a schematic structural diagram showing another organ isolation balloon according to an embodiment of the present disclosure:

FIG. 11 is a schematic structural diagram showing another organ isolation balloon according to an embodiment of the present disclosure:

FIG. 12 is a schematic diagram showing an implementation scenario of a surgery assisting system according to an embodiment of the present disclosure;

FIG. 13 is a schematic diagram showing an implementation scenario of another surgery assisting system according to an embodiment of the present disclosure:

FIG. 14 is a schematic diagram showing a delivering device according to an embodiment of the present disclosure:

FIG. 15 is a top view of a delivery member according to an embodiment of the present disclosure:

FIG. 16 is a front view of a delivery member according to an embodiment of the present disclosure:

FIG. 17 is a side view of a delivery member according to an embodiment of the present disclosure; and

FIG. 18 is a structural block diagram showing a robot according to the present disclosure.

Parts in the figures and reference numerals thereof:

balloon unit 10; chamber 11: airflow control component 12; inflation pipeline 20; delivering device 40; balloon delivery member 41: storage groove 42; delivery conduit 43; first isolation balloon block 101: second isolation balloon block 102: first position 301: second position 302.

### DETAILED DESCRIPTION

To make the objectives, aspects and advantages of the embodiments of the present disclosure become more apparent, a more complete description of the embodiments of the present disclosure will be rendered by reference to the accompanying drawings. It is noted that relational terms such as first and second, and the like, may be used herein to distinguish one entity or action from another entity or action without necessarily requiring or implying any actual such relationship or order between such entities or actions. In the description of the present disclosure, it is to be understood that the terms “central”, “upper”, “lower”, “front”, “rear”, “left”, “right”, “vertical”, “horizontal”, “top”, “bottom”, “inner”, “outer”, and the like designate orientations or positional relationships based on the orientation or positional relationships shown in the figures are merely for convenience in describing the present disclosure and to simplify the description, and do not indicate or imply that the indicated devices or elements must have a particular orientation, be constructed and operated in a particular orientation, and thus should not be construed as limiting the present disclosure. Furthermore, the terms “include”, “include”, or any other variation thereof in this specification are intended to cover a non-exclusive inclusion, such that processes, methods, objects, or apparatuses that include a list of elements include not only those elements, but may also include other elements not expressly listed or inherent to such processes, methods, objects, or apparatuses. Without more limitations, elements defined by the sentence “including one . . . ” does not exclude that there are still other same elements in the processes, methods, objects, or apparatuses. If there is no conflict, embodiments of the present disclosure and various features of the embodiments may be combined with each other within the scope of the present disclosure.

This embodiment is used to reduce the negative impact of radiotherapy or ablation therapy on adjacent organs, i.e., to isolate the target organ during the radiotherapy or ablation therapy process from the surrounding environment to realize the protection of surrounding cavity or adjacent organs during the radiotherapy or ablation therapy process.

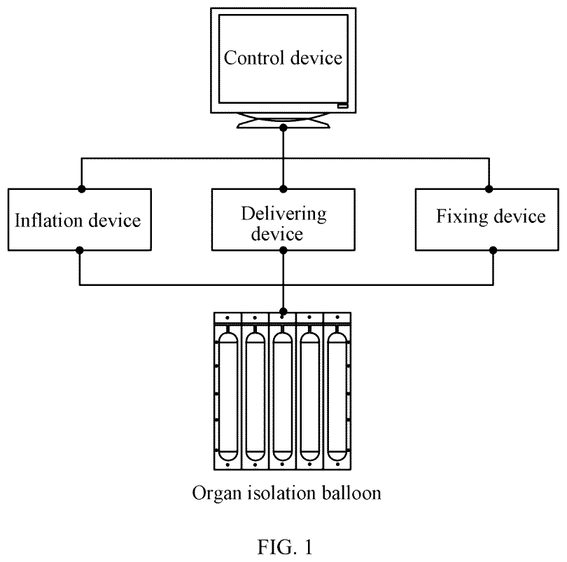

Specifically: this embodiment provides a surgery assisting system, as shown in FIG. 1, which is a schematic diagram showing the framework of a surgery assisting system provided in an embodiment of the present disclosure, and shows that the surgery assisting system includes a control device, an organ isolation balloon, a delivering device, an inflation device and a fixing device. Specifically, the control device is used to control the delivering device, the inflation device, and the fixing device, and to configure and move the organ isolation balloon to achieve collaborative coordination between the devices. It also ensures the adaptability and specific configuration of the organ isolation balloon for the target organ under radiotherapy or ablation therapy, thereby protecting the surrounding cavity or adjacent organs during the radiotherapy or ablation therapy process.

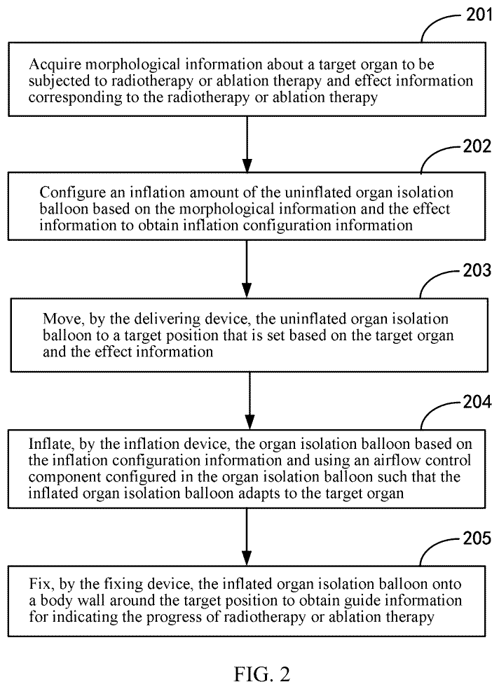

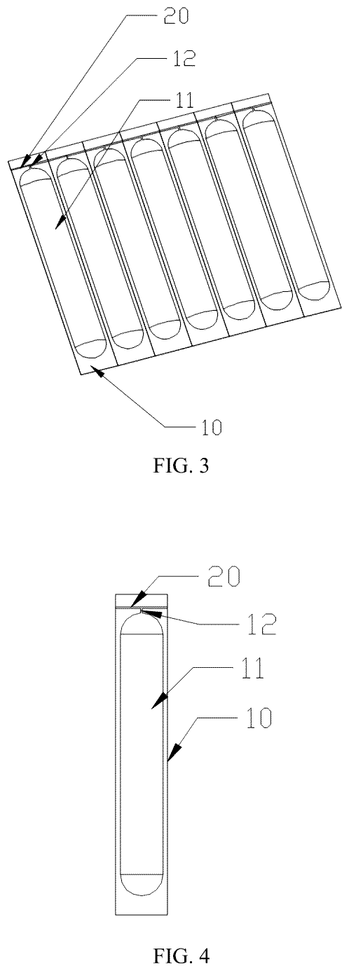

In the following, the control process of the control device for the organ isolation balloon, the delivering device, the inflation device and the fixing device in the surgery assisting system is described, i.e., the following method steps are performed by the control device. The control device may be an intelligent terminal, a dedicated chip configured in the surgery assisting system or other entity with a control function. As shown in FIG. 2, which is a schematic flowchart showing implementation of a surgery assisting system according to an embodiment of the present disclosure, the specific control steps include but are not limited to the following steps.

In a step of 201, morphological information about a target organ to be subjected to radiotherapy or ablation therapy and effect information corresponding to the radiotherapy or ablation therapy are acquired.

In this embodiment, the target organ subjected to the radiotherapy or ablation therapy may be liver, uterus, adrenal gland, lung, prostate, and other sites where tumors may occur. The morphological information about the target organ may be the shape contour of the organ, the position in the body. In addition, the effect information corresponding to the radiotherapy or ablation therapy is therapeutic effect to be achieved by the radiotherapy or ablation therapy, such as the radiation intensity, the number of action and the range of action during the treatment.

Specifically, the morphological information and effect information may be determined through imaging evaluation, i.e., the shape and position of target organ may be determined through CT or MRI data of a patient to determine the fixation position, the isolation distance of the balloon and configure appropriate balloon.

It is to be understood that the morphological information and the effect information may be input and set by a relevant person according to practical experience, or may be recognized based on an image recognition system, for example, automatically recognizing the shape of the target organ and the position of the tumor region through AI, and automatically configuring the corresponding dose.

In a step of 202, an inflation amount of the uninflated organ isolation balloon based on the morphological information and the effect information are configured to obtain inflation configuration information.

In this embodiment, the organ isolation balloon is in an uninflated state when it has not been delivered into the body, and may be in a rolled shape or other shape convenient for delivery. The inflation configuration information is adapted to the shape and position of the target organ and the air volume corresponding to the isolation distance.

In one possible scenario, the organ isolation balloon includes a plurality of balloon units, as shown in FIG. 3, i.e., the organ isolation balloon includes at least two balloon units 10 connected together, the balloon units 10 are each provided with a chamber 11 capable of containing air, and the chambers 11 of the balloon units 10 are not in communication with each other. In addition, each balloon unit 10 is provided with a corresponding airflow control component 12, and the plurality of balloon units 10 are in communication with an inflation pipeline 20 so as to realize the inflation process of the plurality of balloon units.

Specifically, for the structure of an individual unit 10, as shown in FIG. 3, each balloon unit 10 is provided with a corresponding airflow control component 12 for communicating the interior of the chamber 11 with the inflation pipeline 20 when filling the chamber 11 of the balloon unit 10 with air such that the air enters the chamber 11 of the balloon unit 10 from the outside of the chamber 11 of the balloon unit 10 and prevents the air from leaking from the inside to the outside of the chamber 11 of the balloon unit 10.

The organ isolation balloon of this embodiment is composed of a plurality of balloon units 10, where the number of balloon units 10 is two or more. Each balloon unit 10 is provided with a chamber 11 capable of containing air, and the balloon unit 10 can be inflated by filling the chamber 11 of the balloon unit 10 with the air. Since the chambers 11 of the respective balloon units 10 are not communicated with each other, the use of other normal balloon units 10 is not affected even if the individual balloon units 10 are damaged or leaked. This embodiment also provides each balloon unit 10 with an airflow control component 12 that can control communication between the interior of the chamber 11 of each balloon unit 10 and the inflation pipeline 20, and can open airflow passage between the inflation pipeline 20 and the chamber 11 of the balloon unit 10 when it is necessary to inflate the chamber 11 of the balloon unit 10 such that air can enter the chamber 11 of the balloon unit 10 through the inflation pipeline 20. Moreover, the airflow control component 12 has a function of allowing air to enter the chamber 11 of the corresponding balloon unit 10 only in one direction, and not allowing air in the chamber 11 of the balloon unit 10 to leak out of the chamber 11 through the airflow control component 12. Preferably, the airflow control component 12 in this embodiment may employ a check valve which can only be unidirectionally conducted from the inflation pipeline 20 into the chamber 11 of the balloon unit 10, i.e., air can only flow from the inflation pipeline 20 into the chamber 11 of the balloon unit 10 through the check valve and cannot flow out of the chamber 11 of the balloon unit 10 through the check valve.

The shape of the organ isolation balloon of this embodiment may be selected in advance according to the requirements of the treatment. In specific implementation, it is feasible to make imaging evaluation on the patient to be treated, and then select the balloon with different size and/or shape according to the requirements of disease condition. The methods for imaging evaluation include but are not limited to CT, nuclear magnetic resonance and ultrasound imaging. The shape of the selected balloon is such as to completely separate the organ to be treated from other adjacent organs or tissues when the balloon is inflated. Since the organ isolation balloon of this embodiment is composed of a plurality of balloon units 10, the number of balloon units 10 and the shape of each balloon unit 10 may also be set according to the requirement that the organ to be treated can be completely separated from other adjacent organs or tissues in the inflated state of the balloon.

In one possible scenario, this embodiment provides a configurable organ isolation balloon, as shown in FIG. 5, which is a schematic structural diagram showing another organ isolation balloon provided by an embodiment of the present disclosure. The figure shows the airflow direction of the organ isolation balloon during inflation, the air is injected through the air inlet. The path of the air is indicated via the air channel. In order to prevent backflow; a check valve is provided at the connection of each balloon unit and the air channel to prevent backflow. In this way, the air column can be inflated to serve as isolation protection.

Further, the edge of each balloon unit in the organ isolation balloon is seamlessly sealed to prevent air leakage. By using the seamless sealed edge, the balloon units can be separated (cut, pulled or otherwise separated), achieving dynamic configuration for the balloon units. The edge of the balloon unit may also be configured with fixing holes (also serving as a splicing sites), which can be used for the expansion of the balloon unit on the one hand, and for the fixing of the organ isolation balloon on the other hand.

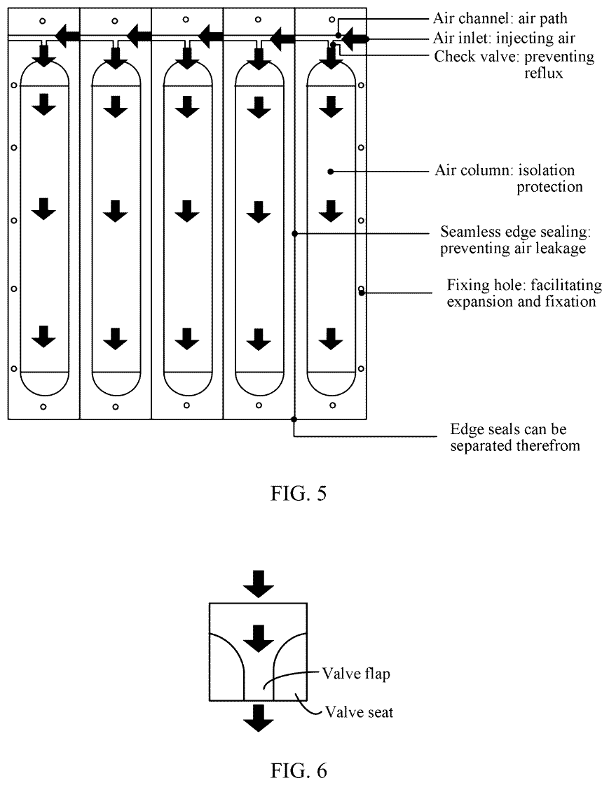

In the above embodiment, the check valve ensures the airtightness of the organ isolation balloon, and the structure of the check valve is described below with reference to FIG. 6, FIG. 6 is a schematic structural diagram showing another check valve provided in an embodiment of the present disclosure. The figure shows the direction in which the air can flow: The check valve refers to a valve that automatically opens and closes the valve flap based on the flow of the air medium itself to prevent the reverse flow of the air. The check valve is automatically operated, and under the action of air pressure flowing in one direction, the valve flap opens: when the air flows in the opposite direction. the combined action of the air pressure and the self-weight of the valve flap on the valve seat cuts off the flow; achieving unidirectional air flow:

In order to match the structural configuration of the organ isolation balloon in the context of the embodiments described above, configuration of the inflation configuration information may be implemented by the control device.

### (1) Configuration of the Number of Balloon Units in the Organ Isolation Balloon.

The acting range information corresponding to the position requiring protection or isolation in radiotherapy or ablation therapy process can be firstly determined based on the morphological information, i.e., the radiation range of the radiotherapy or ablation therapy on the target organ, i.e., the range of the lesion or the range determined based on the range of the lesion. Then the number of balloon units is configured according to the action range information, such that the organ isolation balloon can cover the action range on the target organ corresponding to the action range information, thereby achieving target isolation treatment. The balloon units are connected according to the splicing sites at the edges of the balloon units based on the number of balloon units to obtain an organ isolation balloon. A unit inflation amount of each balloon unit in the organ isolation balloon is configured according to the effect information so as to obtain inflation configuration information, where the unit inflation amount may be an inflation amount in case that the balloon unit is inflated to its full capacity or a self-defined inflation amount.

### (2) Configuration of Isolation Distance for the Organ Isolation Balloon.

Specifically: for the configuration of the isolation distance of the above inflation configuration information, the control device can acquire the effect information acting on the target organ in radiotherapy or ablation therapy process, and determine the acting dose required to reach the effect information, where the acting dose corresponds to a dose when the radiotherapy is about to endanger an adjacent organ or a dose that is capable of protecting an adjacent organ in the ablation therapy. Then an isolation distance is configured for achieving an isolation effect on the target organ according to the acting dose, for example, the greater the acting dose, the greater the configured isolation distance, and the greater the corresponding inflation amount. The unit inflation amount of each balloon unit in the organ isolation balloon is configured based on the isolation distance to obtain inflation configuration information so as to ensure the effect of isolation.

### (3) Air Volume Gradient Configuration for the Organ Isolation Balloon.

It will be appreciated that generally a uniform inflation amount may be applied to the balloon units to adapt to the desired effect of the treatment. However, since the radiation amount in the irradiation center may be greater than that at the edge position in radiotherapy or ablation therapy process, on the basis of satisfying the basic treatment effect, the inflation amount of the balloon unit near the irradiation center may be configured to be high, while the inflation amount of the balloon unit around the irradiation center may be configured to be medium to realize the target organ isolation balloon configuration. This reduces the occupied space of the organ isolation balloon in the body and reduces the compression on the surrounding organs, while ensuring the treatment effect.

It can be understood that the above configuration process of air volume gradient can also further improve the degree of conformity between the organ isolation balloon and the target organ, which takes into account the irregularity of the target organ and reduces the squeezing of the surrounding organ. The gradient can also be configured to be lower at the middle and higher at both sides. The specific gradient depends on the actual scenario.

Specifically, for the scenario where the above inflation configuration information is gradient-based, the control device can acquire effect information acting on the target organ in radiotherapy or ablation therapy process, and determine edge state information corresponding to the effect information on the target organ. Then, based on the edge state information about the target organ, the inflation amount gradient conforming to the target organ is configured, for example, low air volume at the protrusion or high air volume at the lesion. The unit inflation amount of each balloon unit in the organ isolation balloon is determined according to the inflation amount gradient to obtain inflation configuration information.

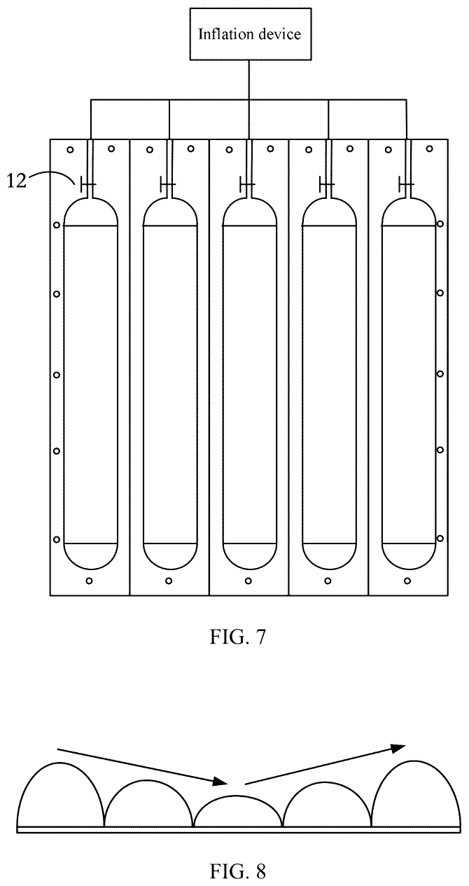

In order to achieve the above gradient inflation process, the structure shown in FIG. 7 can be adopted. As shown in FIG. 7, the airflow control component 12 is arranged at the connecting pipeline of each balloon unit and the inflation device, i.e., the inflation process of each balloon unit is independent from each other such that a self-defined inflation process can be achieved one by one. In one possible scenario, with the structure shown in FIG. 7, a gradient which is high on both sides and low at the middle can be obtained, as shown in FIG. 8, to fit with the protrusion of the organ.

It will be appreciated that the above configurations for the number of organ isolation balloon units, the isolation distance, and the air volume gradient may be used individually, or may be used in combination, for example, an adaptive configuration may be used that takes into account the number of organ isolation balloon units, the isolation distance, and the air volume gradient.

In a step of 203, the uninflated organ isolation balloon is moved by the delivering device to a target position that is set based on the target organ and the effect information.

In this embodiment, in case that the organ isolation balloon is a single individual, it may be delivered directly through the delivering device.

However, the organ isolation balloon in this embodiment may also be composed of a plurality of isolation balloon blocks, i.e., organ isolation balloons configured by custom. In this case, the isolation balloon blocks may be delivered respectively, and the same delivery path may be used. Furthermore, considering the coverage for the target organ, different delivery paths may also be used to inflate and deploy the respective isolation balloon blocks to different positions of the target organ, which are then spliced with each other, thereby improving the coverage.

Specifically, taking an organ isolation balloon including a first isolation balloon block and a second isolation balloon block as an example for explanation. In practical scenarios, more isolation balloon blocks may be included.

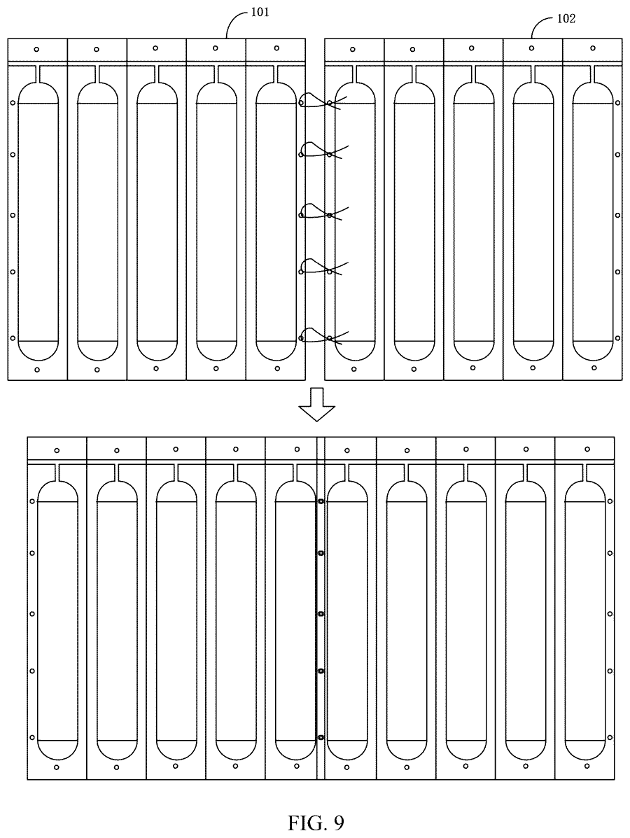

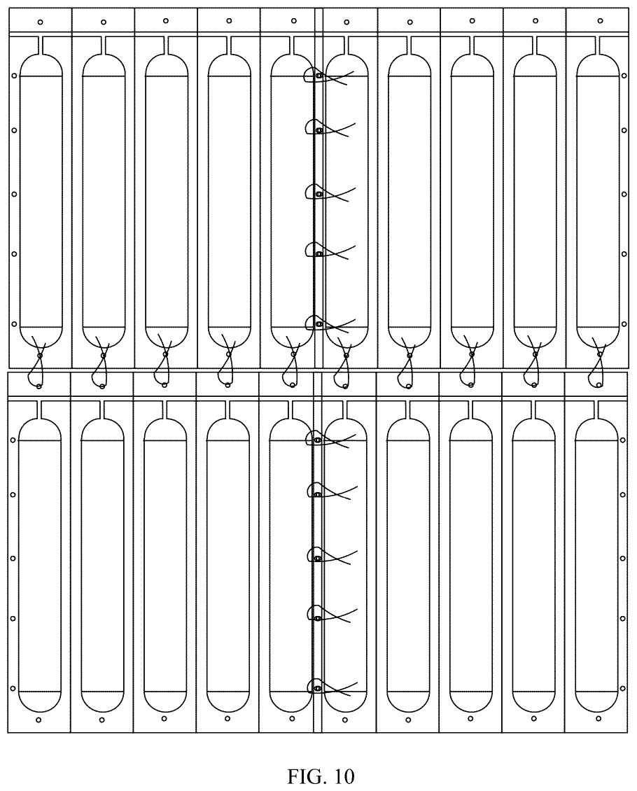

Firstly, splicing information about the first isolation balloon block and the second isolation balloon block corresponding to the organ isolation balloon in radiotherapy or ablation therapy process is determined on the basis of the morphological information, i.e., the splicing edges corresponding to the first isolation balloon block and the second isolation balloon block. As shown in FIG. 9, the splicing edge of the first isolation balloon block 101 is an edge with a splicing site on the right side, and the splicing edge of the second isolation balloon block 102 is an edge with a splicing site on the left side, and the organ isolation balloon with a pre-set shape and size can be obtained by splicing, and the specific splicing methods may be pasting, sewing, etc. Alternatively, the splicing process may involve splicing of multiple edges, for example, as shown in FIG. 10, by splicing the upper and lower edges, and splicing the left and right edges, a larger and more comprehensively wrapped organ isolation balloon can be obtained.

It is to be understood that the organ isolation balloon obtained by splicing is not limited to a planar type, but may also be a three-dimensional type. As shown in FIG. 11, a barrel-shaped organ isolation balloon wraps a target organ in a three-dimensional isolation space, thereby improving the isolation effect.

After splicing, a first position to which the first isolation balloon block needs to be moved and a second position to which the second isolation balloon block needs to be moved can be determined according to a target position configured for the organ isolation balloon and the splicing information, where the second position is associated with the first position via the target position, i.e., ensuring that the first isolation balloon block can be spliced after being deployed at the first position with the 20) second isolation balloon block after being deployed at the second position. Further, the first isolation balloon block and the second isolation balloon block can be moved by the delivering device such that the first isolation balloon block moves to a first position and the second isolation balloon block moves to a second position. Correspondingly, for the process of using the inflation configuration information to inflate via the airflow control unit configured in the organ isolation balloon by the inflation device such that the inflated organ isolation balloon is adapted to the shape of the target organ, the first isolation balloon block and the second isolation balloon block are inflated using the inflation configuration information by the inflation device. Then a first splicing site at an edge of the inflated first isolation balloon block (i.e., the right side edge of the first isolation balloon block 101 in FIG. 9) and a second splicing site at an edge of the inflated second isolation balloon block (i.e., the left side edge of the second isolation balloon block 102 in FIG. 9) are determined according to the splicing information. The edge of the inflated first isolation balloon block and the edge of the inflated second isolation balloon block are spliced based on the first splicing site and the second splicing site such that the inflated first isolation balloon block and the inflated second isolation balloon block can wrap the target organ.

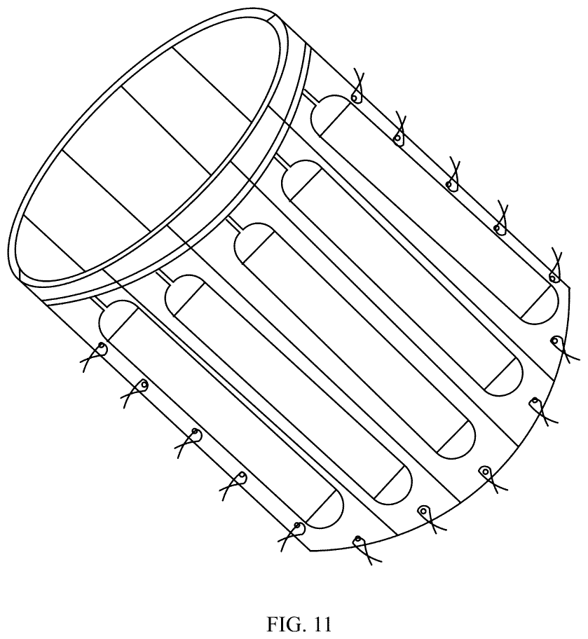

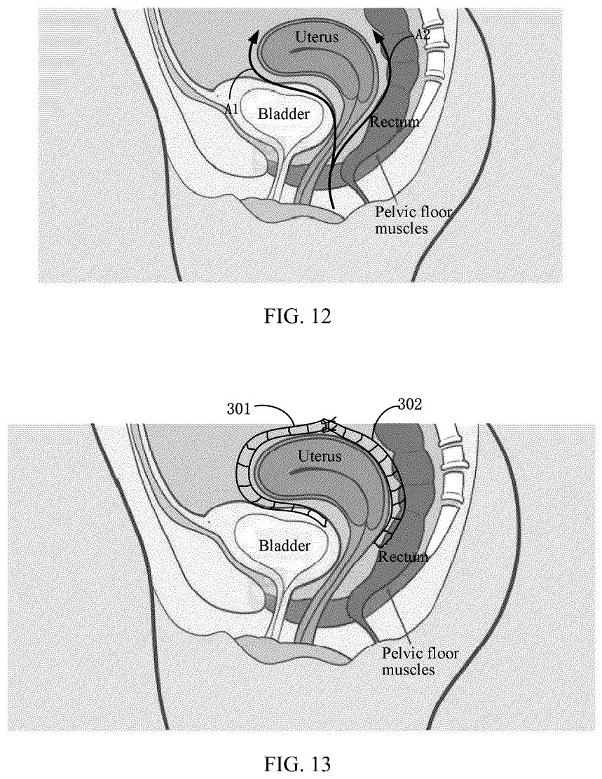

In another possible scenario, it may be necessary to avoid surrounding tissues or organs requiring protection, such as blood vessels, heart, etc, during delivery by the delivering device, so it is necessary to make path planning. As shown in FIG. 12, firstly a target object which is adjacent to the target organ and requires protection is determined. Then a first path A1 is planned for the first isolation balloon block based on a position of the target object such that the first isolation balloon block bypasses the target object, e.g., the bladder, during movement, and the first isolation balloon block is delivered to the first position via a first path through the delivery hole by the delivering device. Then a second path A2 is planned for the second isolation balloon block based on the position of the adjacent organ such that the second isolation balloon block bypasses the target object during movement, and the second isolation balloon block is delivered to the second position via a second path through the delivery hole by the delivering device. The isolation balloon blocks are unfolded and spliced after being moved to the corresponding positions. As shown in FIG. 13, the first isolation balloon block is delivered to the first position 301, and the second isolation balloon block is delivered to the second position 302. The first and second isolation balloon blocks are inflated and spliced such that the influence on the surrounding organ can be reduced and the coverage for the target organ can be improved.

In particular, in the scenario shown in FIG. 13. i.e., when treatment of the uterus is required, the organ isolation balloon blocks are placed at a first position 301 between the uterus and rectum and a second position 302 between the uterus and the bladder according to patient's CT or MRI data, and the bladder and rectum can be pushed away from the uterus when the organ isolation balloon is put in place and inflated. The organ isolation balloon in this embodiment is not inflated with air in the chamber of each balloon unit before being inserted into a pre-set position in the human body, so the organ isolation balloon in this embodiment can be compressed into a smaller volume and then inserted into the human body using the conduit, and the chamber of each balloon unit is inflated with air after the organ isolation balloon reaches the pre-set position in the human body such that the balloon is inflated into a shape capable of completely separating the organ to be treated from other adjacent organs or tissues.

In a step of 204, the organ isolation balloon is inflated by the inflation device with the inflation configuration information via an airflow control component configured in the organ isolation balloon such that the inflated organ isolation balloon adapts to the target organ.

In this embodiment, the airflow control component may be a check valve. i.e., the air cannot escape after filling, thereby ensuring the reliability of the organ isolation balloon.

Specifically, the check valve may include a swing check valve, a lifting check valve or a butterfly check valve. The swing check valve may be a single-flap check valve, a double-flap check valve or a multi-flap check valve. The lifting check valve may be vertical or horizontal. The butterfly check valve may be a micro slowly-closed butterfly check valve, a butterfly buffer check valve, a wafer type double-flap check valve or a micro-resistance slowly-closed butterfly check valve. The specific check valve depends on the actual scenario and is not limited herein.

In a step of 205, the inflated organ isolation balloon is fixed by the fixing device onto a body wall around the target position to obtain guide information for indicating the progress of radiotherapy or ablation therapy.

In this embodiment, since the organ isolation balloon may need to be left in the body to accommodate the need for irradiation once or multiple times, or even more than 20 times, for example, the external irradiation for cervical cancer is usually 25 times, it is necessary to fix the inflated organ isolation balloon.

Specifically, for the methods for fixing by the fixing device, one method is to suture it to the abdominal wall or chest wall. For example, after being placed at the desired position in front of pancreas, the balloon is inflated to isolate the gastrointestinal tract, and the four corners of the balloon is sutured to the abdominal wall. The suture is removed when the treatment is finished.

The other method is to fix it to the body wall through fixed nail (specially used for suture and ligation instrument, which has been applied in clinical practice). When the treatment is finished, the balloon fixing device at the suture site is cut off, and the rest is withdrawn.

In one possible scenario, the fixing device in the surgery system of this embodiment is used to fix the organ isolation balloon to the body wall of the human body. After the organ isolation balloon is placed in the target position and inflated in an inflated state, the organ isolation balloon may be fixed to the body wall of the human body using the fixing device in this embodiment. In the surgery system of this embodiment, after the organ isolation balloon is inflated with the inflation device to its full capacity; the position of the organ isolation balloon in the human body is adjusted laparoscopically with the curved forceps until the organ isolation balloon is located at the desired position, and then the organ isolation balloon is fixed on the body wall of the human body with the fixing device of this embodiment.

Thus, the shape and position of the organ isolation balloon with respect to each organ in the body can remain fixed for a long period of time, and the organ isolation balloon can always be in a shape and position to completely isolate the organ to be treated from adjacent organs or tissues during ablation therapy or radiotherapy, thereby providing more thorough and effective protection for other organs or tissues in the human body during treatment. Moreover, since the surgery system of this embodiment can keep the shape and position of the organ isolation balloon or the combined organ isolation balloon stable, this embodiment can still provide good protection for some organs deep in the human body which cannot be effectively protected by the existing isolation balloon, for example, the surgery system of this embodiment can provide effective protection for vital organs such as stomach and intestine in the radiotherapy for pancreatic cancer.

In this embodiment, the fixing device is a suture, and the surgery system in this embodiment may suture the organ isolation balloon or the combined organ isolation balloon on the abdominal wall or the chest wall using the suture. When the treatment is finished, the suture is removed.

For example, when the surgery system of this embodiment is applied to the radiotherapy for pancreatic cancer, the organ isolation balloon or the combined organ isolation balloon may be placed at a desired position in front of the pancreas, then inflated to isolate organs such as the gastrointestinal tract, and the four corners of the balloon may be sutured to the abdominal wall.

As another implementation, in this embodiment, the fixing device is a fixing frame, such as a laparoscopic clip. In the specific implementation, it is feasible to select different positions and different numbers of fixing clips for fixation according to the requirements for organ or human tissue isolation. When the treatment procedure is completed, the fixing clip is removed from the laparoscope conduit and the skin is sutured.

A possible configuration of the balloon unit 10 will be described below.

In this embodiment, the material used for the balloon unit 10 is: biocompatible materials such as polylactic acid, polypropylene and polyvinyl chloride, and the specific materials are not limited herein.

In this embodiment, the organ isolation balloon is an integrated structure composed of the balloon units 10. Although the organ isolation balloon in this embodiment includes a plurality of balloon units 10, the organ isolation balloon may be integrally formed into an integrated structure, which is convenient to manufacture and does not require connection of the individual balloon units 10.

It is also possible in other embodiments to provide some or all the balloon units 10 of the organ isolation balloon as a separate structure as desired, and then connect these balloon units 10 together to form a complete organ isolation balloon.

In this embodiment, the balloon units 10 are arranged side by side in a first pre-set direction. The structure of the organ isolation balloon formed by arranging the balloon units 10 side by side as described above is simple, and the organ isolation balloon is easily fully deployed after the balloon units 10 are inflated. The first pre-set direction may be selected from a length direction, a width direction, or any other direction of the balloon unit 10, without limitation.

In this embodiment, the airflow control components 12 of the respective balloon units 10 are located at the same end of the organ isolation balloon. In the aforementioned manner, the inflation pipeline can communicate with the airflow control components 12 of the respective balloon units 10 at the same end of the organ isolation balloon, simplifying the arrangement of the inflation pipeline.

This embodiment may also splice the plurality of organ isolation balloons described above into one large organ isolation balloon. Although a single organ isolation balloon may be used in different sizes and shapes depending on the condition, two or more organ isolation balloons may be combined in a splicing manner to obtain a larger organ isolation balloon in case that the single organ isolation balloon fails to meet treatment requirements. In this embodiment, two or more organ isolation balloons may be sutured and fixedly connected to obtain a larger organ isolation balloon to meet the requirements of treatment.

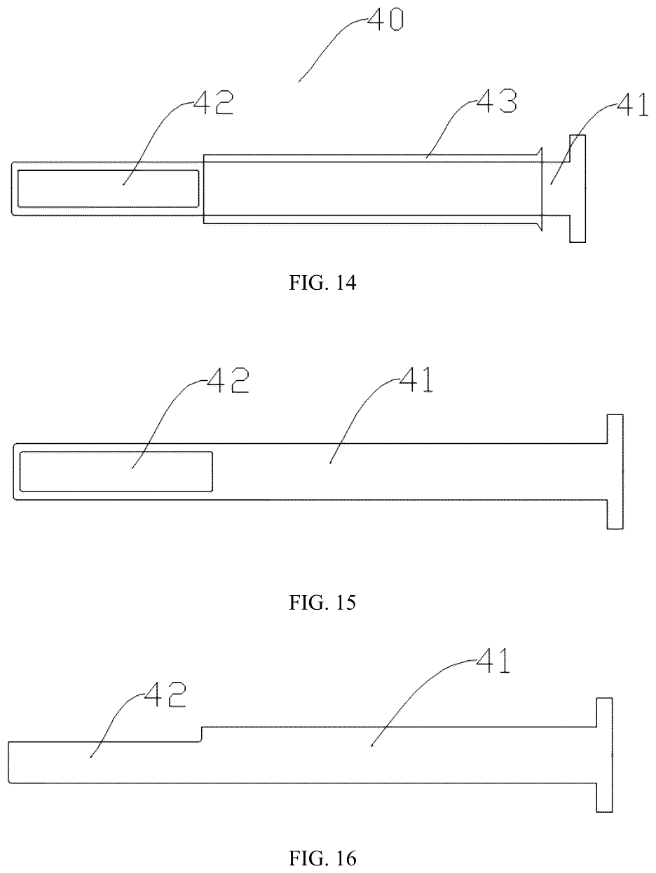

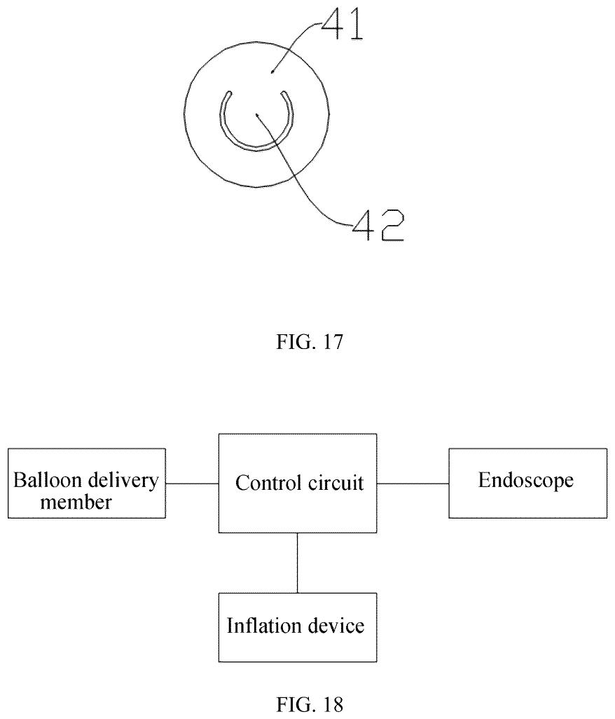

In this embodiment, a delivering device 41 is used to deliver the organ isolation balloon a target position inside the human body from outside. As shown in FIG. 14, the delivering device 41 includes a conduit 43 with a hollow pipeline, and a balloon delivery member provided at one end thereof with a storage groove 42 for storing the organ isolation balloon, as shown in FIGS. 15 and 16, the balloon delivery member is delivered to a target position inside a human body along the pipeline of the conduit 43 and exposes the storage groove 42 to the outside of the conduit. The conduit 43 may be a laparoscope conduit.

Moreover, the surgery assisting system further includes an endoscope and a television monitoring device, the endoscope is configured for acquiring real-time images of an interior of the human body and transmitting the real-time images to the television monitoring device for displaying the morphological information, the inflation configuration information, and the guide information in radiotherapy or ablation therapy process.

Specifically, the endoscope has a light source system, and the endoscope is configured for acquiring a real-time image of the interior of the human body and transmitting the real-time image to the television monitoring device. The endoscope includes but is not limited to a laparoscope and a thoracoscope. The surgery system of this embodiment may use an endoscope to place an organ isolation balloon into a target position within a patient's body under television monitoring.

It is to be understood that, in order to deliver the organ isolation balloon from the outside of the human body to the target position inside the human body using the balloon delivery member, the surgery system of this embodiment further includes a puncture needle having a tip extending from one end of the conduit 43 to the other end of the conduit. A minute wound may be opened on the human body using the puncture needle and then the conduit 43 may be delivered to the inside of the human body through the wound.

In this embodiment, the uninflated organ isolation balloon is folded and placed in the storage groove 42 at the front end of the balloon delivery member, and then the balloon delivery member containing the organ isolation balloon is fed from the outside of the human body into the conduit 43 with the end containing the organ isolation balloon facing forward, and the balloon delivery member is pushed along the pipeline into the interior of the human body, and finally the portion of the balloon delivery member containing the organ isolation balloon is exposed from the conduit 43. In order to facilitate the placement of the organ isolation balloon and the successful deployment of the organ isolation balloon after inflation, the balloon delivery member of this embodiment has a hollow cylindrical structure, and a storage groove 42 for the organ isolation balloon is formed after removing a part of the wall of the hollow cylindrical body at one end of the balloon delivery member. The inflation device of the surgery system in this embodiment includes an air source and an inflation pipeline 20 that communicates the air source with the airflow control component 12 of each balloon unit 10. After the organ isolation balloon is placed in the target position within the human body, the inflation tube is inserted through the endoscopic conduit, and is connected with the inflation tube port such that the inflation tube is in communication with the air source. The inflation device is started, the air from the air source reaches the airflow control component 12 of each balloon unit 10 through the inflation pipeline 20, and then enters the chamber 11 of each balloon unit 10 through the respective airflow control component 12.

The above embodiments describe the control flow for the surgery assisting system and the possible configurations of the organ isolation balloon. The operation process of the surgery assisting system is described below in combination with the specific flow.

In a step of S1, the position where the organ isolation balloon of the present disclosure is placed and the fixation position and the isolation distance between the adjacent organ or device and the organ to be treated are determined through imaging evaluation.

In a step of S2, a suitable organ isolation balloon or a combined organ isolation balloon is selected according to the position where the organ isolation balloon of the present disclosure is placed and the fixation position and the isolation distance between the adjacent organ or device and the organ to be treated.

The above two steps can determine the placement position and the fixation position for the organ isolation balloon and the isolation distance according to the CT or MRI data of the patient to select a suitable organ isolation balloon.

In a step of S3, under television monitoring, the organ isolation balloon is placed in a pre-set position using endoscopic technology.

This step allows for laparoscopic placement of the organ isolation balloon to the desired position in the operating room under television monitoring. The position of the organ isolation balloon is then adjusted laparoscopically with curved forceps until the organ isolation balloon is adjusted to the desired position.

In a step of S4, the chambers 11 of the balloon units 10 are inflated one by one using an inflation device to inflate the organ isolation balloon.

In this step, the inflation tube can be sent into the body through the laparoscope conduit, and an inflation tube port is connected to inflate the organ isolation balloon.

In a step of S5, the organ isolation balloon is fixed with the patient's abdominal wall or chest wall using a fixing device after completing the inflation.

In this step, the organ isolation balloon is fixed to the patient's abdominal wall or chest wall via a fixing clip, such as a laparoscopic clip. In the specific operation, the organ isolation balloon can be fixed at different positions according to the isolation requirements and fixed with different numbers of fixing clips.

In a step of S6, ablation therapy or radiotherapy is performed for the target organ.

In this step, CT or MRI scanning may be performed again for the corresponding part of the patient to determine the tumor isolation effect, the tumor target area may be delineated in the treatment planning system, and the physicist can make a radiotherapy or ablation therapy plan, and the radiotherapy or ablation therapy technician can perform the radiotherapy or ablation therapy plan. In the specific implementation, the irradiation may be performed once or multiple times, or even more than 20 times, for example, the external irradiation for cervical cancer is usually 25 times.

In a step of S8, the organ isolation balloon is pierced.

In this step, laparoscope surgery is performed again after the treatment is completed, and a scalpel is sent into the body through a laparoscope conduit, and then the organ isolation balloon is pierced sequentially.

In a step of S9, the fixing device is released.

In this step, the fixing device is removed after piercing the organ isolation balloon to release the connection between the organ isolation balloon and the human body wall.

In a step of S10, the organ isolation balloon or the combined organ isolation balloon is removed from the patient using endoscopic techniques.

In this step, after releasing the fixing device, the balloon is withdrawn along the laparoscope conduit.

In a step of S11, at the end of the surgery; the laparoscope is removed and the skin is sutured.

Compared to the existing technology, the surgery system of the present disclosure has a wider range of applications and can be applied to radiotherapy in addition to ablation therapy. The organ isolation balloon or the combined organ isolation balloon of the present disclosure can be fixed to the abdominal wall or chest wall of the human body using a fixing device such that the position of the balloon can be kept fixed during treatment without displacement. In addition, the duration for keeping the fixation can be adapted to the needs of patients without limitation.

In another possible scenario, an embodiment of the present disclosure further provides a surgical robot, including a control circuit (which may be the execution circuit of the control device in the above embodiment), a manipulator and the surgery assisting system according to the above embodiment. As shown in FIG. 18, the control circuit is respectively electrically connected to the endoscope system and the control circuit, and the control circuit is configured for receiving a real-time image of an interior of the human body captured by the endoscope. The control circuit is configured for controlling the manipulator to convey the balloon delivery member with the organ isolation balloon along the pipeline of the conduit 43 to a target position inside the human body according to the real-time images.

In this embodiment, the manipulator of the surgical robot may be used to feed the balloon delivery member with the organ isolation balloon into the endoscope and push the balloon delivery member to be delivered along the pipeline of the conduit 43 from the inside of the human body to the outside of the human body, and the control circuit may acquire the real-time image captured by the endoscope during the balloon delivery by the balloon delivery member and analyze the acquired images, and stop delivery when the end of the balloon delivery member with the organ isolation balloon is completely exposed out of the conduit 43 and is at the target position.

In this embodiment, the control circuit is electrically connected to the inflation device, and the control circuit is further configured to control the inflation device to inflate the balloon after the balloon delivery member is delivered to the target position within the human body. When the control circuit determines that the balloon delivery member has been delivered in place according to the real-time images collected by the endoscope, a control signal is sent to the inflation device to control the inflation device to start to inflate the balloon, and when the inflation is completed, the control circuit controls the inflation device to turn off.

The control circuit in the embodiments described above may include a processor and a memory storing computer program instructions.

In particular, the processor described above may be a Central Processing Unit (CPU), may be other general-purpose processor, Digital Signal Processor (DSP), Application Specific Integrated Circuit (ASIC), Field-Programmable Gate Array Gate Array (FPGA) or other programmable logic device, discrete gate or transistor logic device, discrete hardware components, etc. and may be one or more integrated circuits configured to implement embodiments of the present disclosure.

The memory may include, among other things, mass storage for data or instructions. By way of example, and not limitation, the memory may include a Hard Disk Drive (HDD), a floppy disk drive, a flash memory; an optical disk, a magneto-optical disk, a magnetic tape, or a Universal Serial Bus (USB) drive, or a combination of two or more of the foregoing. Where appropriate, the memory may include removable or non-removable (or fixed) media. Where appropriate, the memory may be internal or external to the data processing apparatus. In a particular embodiment, the memory is a non-volatile solid-state memory. In a particular embodiment, the memory includes a read only memory (ROM). Where appropriate, the ROM may be a mask programmed ROM, a programmable ROM (PROM), an erasable PROM (EPROM), an electrically erasable PROM (EEPROM), an electrically alterable ROM (EAROM) or a flash memory or a combination of two or more of the above.

The above only describes the specific embodiments of the present disclosure. It will be apparent to a person of ordinary skill in the art that the specific operations of the systems, modules and units described above may be referred to corresponding processes in the foregoing method embodiments for convenience and brevity of description and will not be described in detail herein. It should be understood that the scope of the present disclosure is not limited thereto, and that various equivalent modifications or substitutions within the technical scope of the present disclosure will be readily conceivable by a person of ordinary skill in the art, and these modifications or substitutions shall all fall within the scope of the present disclosure.

1. A surgery assisting system in radiotherapy or ablation therapy process,
comprising a control device, an organ isolation balloon, a delivering device, an inflation device and a fixing device, the control device being configured for:
acquiring morphological information about a target organ to be subjected to radiotherapy or ablation therapy and effect information corresponding to the radiotherapy or ablation therapy;
configuring an inflation amount of the uninflated organ isolation balloon based on the morphological information and the effect information to obtain inflation configuration information;
moving, by the delivering device, the uninflated organ isolation balloon to a target position that is set based on the target organ and the effect information;
inflating, by the inflation device, the organ isolation balloon based on the inflation configuration information and using an airflow control component configured in the organ isolation balloon, such that the inflated organ isolation balloon adapts to the target organ; and
fixing, by the fixing device, the inflated organ isolation balloon onto a body wall around the target position to obtain guide information for indicating progress of the radiotherapy or ablation therapy.

2. The surgery assisting system according to claim 1, wherein the organ isolation balloon comprises a plurality of balloon units, and the configuring an inflation amount of the uninflated organ isolation balloon based on the morphological information and the effect information to obtain inflation configuration information comprises:
determining acting range information corresponding to a position requiring protection or isolation in radiotherapy or ablation therapy process based on the morphological information;
configuring a quantity of the balloon units according to the acting range information corresponding to a position requiring protection or isolation such that the organ isolation balloon covers an acting range on the target organ corresponding to the acting range information;
connecting the balloon units according to positions of splicing sites at edges of the balloon units based on the quantity of the balloon units to obtain the organ isolation balloon; and
configuring a unit inflation amount of each of the balloon units in the organ isolation balloon according to the effect information to obtain the inflation configuration information.

3. The surgery assisting system according to claim 2, wherein the configuring a unit inflation amount of each of the balloon units in the organ isolation balloon according to the effect information to obtain the inflation configuration information comprises:
acquiring the effect information acting on the target organ in radiotherapy or ablation therapy process, and determining an acting dose required to reach the effect information, wherein the acting dose corresponds to a dose when the radiotherapy is about to endanger an adjacent organ or a dose that is capable of protecting an adjacent organ in the ablation therapy;
configuring an isolation distance for achieving an isolation effect on the target organ according to the acting dose; and
configuring the unit inflation amount of each of the balloon units in the organ isolation balloon based on the isolation distance to obtain the inflation configuration information.

4. The surgery assisting system according to claim 2, wherein the configuring a unit inflation amount of each of the balloon units in the organ isolation balloon according to the effect information to obtain the inflation configuration information comprises:
acquiring the effect information acting on the target organ in radiotherapy or ablation therapy process, and determining edge state information corresponding to the effect information on the target organ;
configuring an inflation amount gradient attached to the target organ based on the edge state information about the target organ; and
determining the unit inflation amount of each of the balloon units in the organ isolation balloon according to the inflation amount gradient to obtain the inflation configuration information.

5. The surgery assisting system according to claim 1, wherein the organ isolation balloon comprises a first isolation balloon block and a second isolation balloon block,
the moving, by the delivering device, the uninflated organ isolation balloon to a target position comprises:
determining splicing information about the first isolation balloon block and the second isolation balloon block corresponding to the organ isolation balloon in radiotherapy or ablation therapy process based on the morphological information; and
determining a first position to which the first isolation balloon block needs to be moved and a second position to which the second isolation balloon block needs to be moved according to a target position configured for the organ isolation balloon and the splicing information, wherein the second position is associated with the first position through the target position;
moving, by the delivering device, the first balloon block and the second isolation balloon block such that the first isolation balloon block moves to the first position and the second isolation balloon block moves to the second position;
accordingly, the inflating, by the inflation device, based on the inflation configuration information and using an airflow control unit configured in the organ isolation balloon such that the inflated organ isolation balloon adapts to the target organ comprises:
inflating, by the inflation device, the first isolation balloon block and the second isolation balloon block using the inflation configuration information;
determining a first splicing site at an edge of the inflated first isolation balloon block and a second splicing site at an edge of the inflated second isolation balloon block according to the splicing information; and
splicing the edge of the inflated first isolation balloon block and the edge of the inflated second isolation balloon block based on the first splicing site and the second splicing site such that the inflated first isolation balloon block and the inflated second isolation balloon block wrap the target organ.

6. The surgery assisting system according to claim 5, wherein the moving, by the delivering device, the first isolation balloon block and the second isolation balloon block such that the first isolation balloon block moves to the first position and the second isolation balloon block moves to the second position comprises:
determining an adjacent target object corresponding to the target organ and requiring protection;
planning a first path for the first isolation balloon block based on a position of the target object such that the first isolation balloon block bypasses the target object during movement; and
delivering, by the delivering device, the first isolation balloon block to the first position using the first path through a delivery hole; and
planning a second path for the second isolation balloon block based on the position of the adjacent organ such that the second isolation balloon block bypasses a target object during movement; and
delivering, by the delivering device, the second isolation balloon block to the second position using the second path through the delivery hole.

7. The surgery assisting system according to claim 1, wherein the delivering device comprises a delivery conduit and a balloon delivery member;
the delivery conduit is provided with a hollow pipeline, the balloon delivery member is provided with a storage groove for storing the organ isolation balloon at one end of the balloon delivery member, the balloon delivery member is delivered to the target position inside a human body along the pipeline of the delivery conduit, and the storage groove is exposed out of the delivery conduit; and
the delivery conduit is configured for delivering a puncture needle, a tip of the puncture needle penetrates from one end of the delivery conduit to pierce the inflated organ isolation balloon and penetrates from the other end of the delivery conduit, and the pierced organ isolation balloon is taken out of the delivery conduit.

8. The surgery assisting system according to claim 1, wherein the organ isolation balloon in the surgery assisting system comprises a plurality of balloon units, each of the balloon units is provided with a corresponding airflow control component for communicating an interior of a chamber of the respective balloon unit with the inflation device for providing air when filling the air into the chamber, such that the air passes from outside of the chamber of the balloon unit into the chamber of the balloon unit and is prevented from leaking out of the chamber of the balloon unit.

9. The surgery assisting system according to claim 1, further comprising an endoscope and a television monitoring device, the endoscope being configured for acquiring real-time images of an interior of the human body and transmitting the real-time images to the television monitoring device, and the endoscope being configured for displaying the morphological information, the inflation configuration information, and the guide information in radiotherapy or ablation therapy process.

10. A surgical robot, comprising a control circuit, a manipulator and the surgery assisting system according to claim 1, the control circuit being electrically connected to the endoscope and the control circuit, respectively, wherein the control circuit is configured for receiving real-time images of an interior of a human body captured by the endoscope, controlling the manipulator to convey a balloon delivery member with the organ isolation balloon along a pipeline of a delivery conduit to a target position inside the human body according to the real-time images, and controlling the inflation device to inflate the balloon after the balloon delivery member is delivered to the target position within the human body.
